(19) United States
(12) Patent Application Publication
(10) Pub. No.: US 2013/0203151 A1
(43) Pub. Date: Aug. 8, 2013

(54) PRODUCTION OF ALPHAVIRUS REPLICON PARTICLES IN PACKAGING CELLS

(71) Applicants: Scott Balsitis, Cambridge, MA (US); Luis Brito, Cambridge, MA (US); Peter Mason, Cambridge, MA (US)

(72) Inventors: Scott Balsitis, Cambridge, MA (US); Luis Brito, Cambridge, MA (US); Peter Mason, Cambridge, MA (US)

(21) Appl. No.: 13/643,503

(22) PCT Filed: Apr. 26, 2011
(86) PCT No.: PCT/US11/33997

§ 371 Date: Feb. 6, 2013

Related U.S. Application Data
(60) Provisional application No. 61/327,933, filed on Apr. 26, 2010.

(51) Int. Cl. C12N 7/02

(52) U.S. Cl. CPC C12N 7/025
USPC 435/235.1

(57) ABSTRACT

Improvements in packaging cell systems for the high level production of recombinant virus replicon particles useful for directing expression of one or more heterologous gene products.

This application claims the benefit of and incorporates by reference Ser. No. 61/327,933 filed on Apr. 26, 2010.

This project was funded in whole or in part with Federal funds from the National Institute of Allergy and Infectious Diseases, National Institutes of Health, Department of Health and Human Services, under Contract No. HHSN266200500007C, ADB No. N01-AI-50007. The contents of the publication do not necessarily reflect the views or policies of the Department of Health and Human Services, nor does mention of trade names, commercial products, or organizations imply endorsement by the U.S. government.

### FIELD OF THE INVENTION

This invention is in the field of recombinant DNA technology.

### SUMMARY OF THE INVENTION

There is disclosed herein a method of producing virus replicon particles, comprising culturing a packaging cell under conditions suitable for production of virus replicon particles, wherein the conditions comprise a temperature in a range of about 30° C. and about 35° C., wherein the packaging cell comprises: (a) one or more virus structural protein expression cassettes directing expression of virus structural proteins; and (b) a vector selected from the group consisting of a virus vector construct, an RNA vector replicon, a DNA replicon plasmid, a eukaryotic layered vector initiation system, and a virus vector particle, whereby virus vector particles are produced.

There is also disclosed herein a method of producing alphavirus replicon particles, comprising culturing a packaging cell under conditions suitable for production of alphavirus replicon particles, wherein the conditions comprise a temperature in a range of about 30° C. and about 35° C., wherein the packaging cell comprises: (a) one or more alphavirus structural protein expression cassettes directing expression of alphavirus structural proteins; and (b) a vector selected from the group consisting of an alphavirus vector construct, an RNA vector replicon, a DNA replicon plasmid, a eukaryotic layered vector initiation system, and an alphavirus vector particle, whereby alphavirus vector particles are produced.

There is also disclosed herein a method of producing virus replicon particles, comprising a step of culturing a packaging cell under conditions suitable for production of virus replicon particles, wherein the conditions comprise a temperature in a range of about 30° C. and about 35° C., whereby virus replicon particles are produced.

There is also disclosed herein a method of producing alphavirus replicon particles, comprising a step of culturing a packaging cell under conditions suitable for production of alphavirus replicon particles, wherein the conditions comprise a temperature in a range of about 30° C. and about 35° C., whereby alphavirus replicon particles are produced.

There is also disclosed herein a method of producing virus replicon particles, comprising:

(a) introducing into a packaging cell by transfection a virus vector; (b) culturing the packaging cell under conditions suitable for production of virus replicon particles, whereby virus vector particles are produced.

There is also disclosed herein a method of producing alphavirus replicon particles, comprising: (a) introducing into a packaging cell by transfection an alphavirus vector; (b) culturing the packaging cell under conditions suitable for production of alphavirus replicon particles, whereby alphavirus vector particles are produced.

These methods are described in detail below, with reference to the accompanying drawings.

### BRIEF DESCRIPTION OF THE DRAWINGS

Abbreviations used in the descriptions of the drawings are explained in the detailed description.

FIGS. 1A and 1B. Graphs demonstrating that the encoded antigen affects VRP yields. Yields of VRP from BHK cells electroporated with helper RNAs and the indicated replicon were determined 24 hours post-electroporation (FIG. 1A). Yields of VRP from packaging cells transfected with the indicated replicons using DOTAP:DOPE were determined at 48 hours post-transfection (FIG. 1B).

FIG. 2. Graph demonstrating that reduced temperature improves yield of VRPs encoding vaccine-relevant antigens from packaging cells.

FIG. 3. Graph demonstrating that reduced temperature improves VRP yields in transfection-based VRP production from packaging cells.

FIGS. 4A-4C. Comparison of VRP production methods. FIG. 4A, triple-electroporation of susceptible cells (e.g., Vero, BHK); FIG. 4B, replicon electroporation into packaging cells followed by amplification; FIG. 4C, transfection of replicon RNA or replicon plasmid DNA into packaging cells.

FIG. 5. Graph demonstrating VRP production by replicon transfection in packaging cells. Adherent packaging cells were transfected with the indicated replicon RNA at 2 μg RNA per 10⁶ cells using DOTAP:DOPE lipoplexes. Transfection complexes were removed after 4 hours and cells were given fresh medium. Output VRP titers were measured at 24 and 48 hours post-infection.

FIG. 6. Graph demonstrating high VRP yields following PEI-mediated transfection of packaging cells. Adherent packaging cells were transfected with VEE/SIN GFP replicon RNA at 2 μg per 10⁶ cells using the indicated transfection reagents, and output VRP titers were determined 24 and 48 hours later. PEI variants were 25 kD linear PEI (linear 25 k), 2.5 kD linear PEI (linear 2.5 k), and 10 kD branched PEI (branched 10 k). PEI transfections were performed at N:P ratios of 5:1 and 10:1 for each PEI variant, as indicated. DOTAP:DOPE lipoplexes at 4:1 N:P ratio were used as a control.

FIG. 7. Graph showing results of RNA transfection-mediated VRP production in suspension. Suspension packaging cells were transfected with VEE/SIN replicon RNA encoding GFP using DOTAP:DOPE as the transfection reagent and 2 μg RNA per 10⁶ cells. Output VRP titers were determined at 24 and 48 hours post-infection.

FIG. 8. Graph showing results of DNA-launched VRP production in packaging cells. Suspension packaging cells were transfected with the indicated amounts of DNA replicon plasmid using DOTAP:DOPE lipoplexes at a 4:1 N:P ratio. GFP was the replicon-encoded antigen. Supernatants were harvested and titered for GFP expression at the indicated time points.

FIG. 9. Nucleotide sequence of DNA plasmid CMV-TC83CR-GFP (SEQ ID NO:1).

### DETAILED DESCRIPTION

Improved methods of producing virus replicon particles in vitro are described in sections I-III, below. The methods are useful for producing virus replicon particles for any positive-stranded RNA virus, including, but not limited to:
a. Nidovirales, including
   i. Arteriviridae,
   ii. Coronaviridae (e.g., Coronavirus, SARS), and
   iii. Roniviridae;
b. Picornavirales, including
   i. Dicistroviridae,
   ii. Iflaviridae (e.g., infectious flacherie virus),
   iii. Marnaviridae,
   iv. Picornaviridae, (e.g., Poliovirus, the common cold virus, Hepatitis A virus),
   v. Secoviridae (including Comovirinae)
c. Tymovirales, including:
   i. Alphaflexiviridae,
   ii. Betaflexiviridae,
   iii. Gammaflexiviridae,
   iv. Tymoviridae
d. Astroviridae;
e. Barnaviridae;
f. Bromoviridae;
g. Caliciviridae (including Norwalk virus);
h. Closteroviridae;
i. Flaviviridae (e.g., Yellow fever virus, West Nile virus, Hepatitis C virus, Dengue fever virus, Pestiviruses, Bovine Viral Diarrhea virus, and Classical Swine Fever virus, Gadgets Gully virus, Kyasanur Forest disease virus, Langat virus, including the British, Irish, Louping ill, Spanish and Turkish subtypes, Omsk hemorrhagic fever virus, Powassan virus, Karshi virus, Royal Farm virus, Tick-borne encephalitis virus, including the European, Far Eastern, and Siberian subtypes, Kadam virus, Meaban virus, Saumarez Reef virus, Tyuleniy virus, Aroa virus, Bussuquara virus, Iguape virus, Naranjal virus, Dengue virus 1, Dengue virus 2, Dengue virus 3, Dengue virus 4, Kedougou virus, Cacipacore virus, Japanese encephalitis virus, Koutango virus, Alfuy virus, Murray Valley encephalitis virus, St. Louis encephalitis virus, Usutu virus, Kunjin virus, West Nile virus, Yaounde virus, Kokobera virus, Stratford virus, Bagaza virus, Ilheus virus, Rocio virus, Israel turkey meningoencephalomyelitis virus, Ntaya virus, Tembusu virus, Spondweni virus, Zika virus, Banzi virus, Bouboui virus, Edge Hill virus, Jugra virus, Potiskum virus, Saboya virus, Sepik virus, Uganda S virus, Wesselsbron virus, Yellow fever virus, Entebbe bat virus, Sokoluk virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Batu Cave virus, Phnom Penh bat virus, Rio Bravo virus, Cell fusing agent virus, Tamana bat virus, Border disease virus—BD31, Border disease virus—X818, Bovine viral diarrhea virus 1-CP7, Bovine viral diarrhea virus 1-NADL, Bovine viral diarrhea virus 1-Osloss, Bovine viral diarrhea virus 1-SD1, Bovine viral diarrhea virus 2-C413, Bovine viral diarrhea virus 2-New York '93, Bovine viral diarrhea virus 2-strain 890, Classical swine fever virusAlfort/187, Classical swine fever virus—Alfort-Tubingen, Classical swine fever virus—Brescia, Classical swine fever virus—C, Pestivirus of giraffe, Hepatitis C virus, including genotype 10, genotype 11, genotype 1a, genotype 1b, genotype 2a, genotype 2b, genotype 3a, genotype 4a, genotype 5a, genotype 6a, and GB virus B, GB virus A, GB virus C, and Hepatitis G virus-1);
j. Leviviridae;
k. Luteoviridae (e.g.; Barley yellow dwarf virus); Family
l. Narnaviridae;
m. Nodaviridae;
n. Potyviridae;
o. Tetraviridae;
p. Togaviridae (e.g., Rubella virus, Alphaviruses);
q. Tombusviridae;
r. Benyvirus;
s. Furovirus;
t. Hepevirus (e.g., Hepatitis E virus);
u. Hordeivirus;
v. Idaeovirus;
w. Ourmiavirus;
x. Pecluvirus;
y. Pomovirus;
z. Sobemovirus;
aa. Tobamovirus (e.g., tobacco mosaic virus);
bb. Tobravirus;
cc. Tricornavirus;
dd. Umbravirus

In some embodiments the virus replicon particles produced are alphavirus replicon particles. As used herein, the term “alphavirus” has its conventional meaning in the art and includes various species such as Venezuelan Equine Encephalitis virus (VEE; e.g., Trinidad donkey, TC83CR, etc.), Semliki Forest virus (SFV), Sindbis, Ross River Virus, Western Equine Encephalitis Virus, Eastern Equine Encephalitis Virus, Chikungunya, S.A. AR86, Everglades virus, Mucambo, Barmah Forest Virus, Middelburg Virus, Pixuna Virus, O′ nyong-nyong Virus, Getah Virus, Sagiyama Virus, Bebaru Virus, Mayaro Virus, Una Virus, Aura Virus, Whataroa Virus, Banbanki Virus, Kyzylagach Virus, Highlands J Virus, Fort Morgan Virus, Ndumu Virus, and Buggy Creek Virus.

A virus replicon particle (VRP) or “replicon particle”, e.g., an “alphavirus replicon particle,” is a virus (e.g., alphavirus) replicon packaged with virus (e.g., alphavirus) structural proteins. The “virus replicon” or “replicon” (e.g., “alphavirus replicon”) is an RNA molecule which can direct its own amplification in an appropriate target cell. The alphavirus replicon encodes the polymerase(s) which catalyze RNA amplification (nsP1, nsP2, nsP3, nsP4) and contains cis-acting RNA sequences required for replication which are recognized and utilized by the encoded polymerase(s). An alphavirus replicon typically contains the following ordered elements: 5′ viral sequences required in cis for replication, sequences which encode biologically active alphavirus nonstructural proteins (nsP1, nsP2, nsP3, nsP4), 3′ viral sequences required in cis for replication, and a polyadenylate tract. The alphavirus RNA vector replicon also may contain one or more viral subgenomic “junction region” promoters directing the expression of one or more heterologous nucleotide sequence(s). The junction region promoter(s) may, in certain embodiments, be modified in order to increase or reduce viral transcription of the subgenomic fragment and heterologous sequence(s) to be expressed.

In some embodiments an alphavirus replicon is a chimeric replicon, such as a VEE-Sindbis chimeric replicon (VCR) or TC83-Sindbis chimeric replicon (TC83CR). In some embodiments a VCR contains the packaging signal and 3′ UTR from a Sindbis replicon in place of sequences in nsP3 and at the 3′ end of a VEE replicon; see Perri et al., J. Virol. 77, 10394-403, 2003. In some embodiments, a TC83CR contains the packaging signal and 3′ UTR from a Sindbis replicon in place of sequences in nsP3 and at the 3′ end of the TC83CR replicon. Chimeric alphavirus replicons are useful in the production of chimeric alphavirus particles in which one or more of the alphavirus structural proteins is from an alphavirus different to the alphavirus from which at least a part of the replicon is derived.

A virus (e.g., alphavirus) replicon particle containing a virus (e.g., alphavirus) replicon encoding an exogenous protein can be used as a gene delivery vehicle (also referred to herein as “virus vector,” e.g., an “alphavirus vector”) and is particularly useful for delivering to cells in vivo antigens that can raise an immune response. One advantage of such vectors, described in more detail below, is that the encoded antigen can be changed simply by changing one or more polynucleotide cassettes placed under the control of a subgenomic promoter. Changing the antigen does not alter the structure of the VRPs produced, and thus the VRP production process may be similar for a variety of antigens.

Replicons (e.g., alphavirus replicons) encoding an exogenous protein of interest can be assembled into a VRP using a packaging cell. The packaging cell, described in more detail below, contains one or more different virus (e.g., alphavirus) structural protein cassettes which provide the virus (e.g., alphavirus) structural proteins. An “alphavirus structural protein cassette” is an expression cassette that encodes one or more alphavirus structural proteins and which optionally comprises at least one and preferably five copies of an alphavirus replicase recognition sequence. Structural protein expression cassettes typically comprise, from 5′ to 3′ the following ordered elements: a 5′ sequence which initiates transcription of alphavirus RNA, an optional alphavirus subgenomic region promoter, a nucleotide sequence encoding the alphavirus structural protein, a 3′ untranslated region (which also directs RNA transcription and typically contains the one or more copies of an alphavirus replication recognition sequence), and a polyA tract. See WO 2010/019437.

In preferred embodiments two different alphavirus structural protein cassettes (“split” defective helpers) are used in a packaging cell to minimize recombination events which could produce a replication-competent virus. In some embodiments an alphavirus structural protein cassette encodes the capsid protein (C) but not either of the glycoproteins (E2 and E1). In some embodiments an alphavirus structural protein cassette encodes the capsid protein and either the E1 glycoprotein or the E2 glycoprotein (but not both). In some embodiments an alphavirus structural protein cassette encodes the E2 and E1 glycoproteins but not the capsid protein. In some embodiments an alphavirus structural protein cassette encodes the E1 glycoprotein or the E2 glycoprotein (but not both) and not the capsid protein.

A “packaging cell” is a cell that contains one or more virus structural protein expression cassettes and that produces recombinant virus. A packaging cell may be a mammalian cell or a non-mammalian cell, such as an insect (e.g., SF9) or avian cell (e.g., a primary chick or duck fibroblast or fibroblast cell line). See U.S. Pat. No. 7,445,924. Avian sources of cells include, but are not limited to, avian embryonic stem cells such as EB66® (VIVALIS); chicken cells, including chicken embryonic stem cells such as EBx® cells, chicken embryonic fibroblasts, and chicken embryonic germ cells; duck cells such as the AGE1.CR and AGEECR.pIX cell lines (ProBioGen) which are described, for example, in Vaccine 27:4975-4982 (2009) and WO2005/042728); and geese cells. In some embodiments, a packaging cell is a primary duck fibroblast or duck retinal cell line, such as AGE.CR(PROBIOGEN).

Mammalian sources of cells include, but are not limited to, human or non-human primate cells, including PerC6 (PER.C6) cells (CRUCELL N.V.), which are described, for example, in WO 01/38362 and WO 02/40665, as well as deposited under ECACC deposit number 96022940); MRC-5 (ATCC CCL-171); WI-38 (ATCC CCL-75); fetal rhesus lung cells (ATCC CL-160); human embryonic kidney cells (e.g., 293 cells, typically transformed by sheared adenovirus type 5 DNA); VERO cells from monkey kidneys); cells of horse, cow (e.g., MDBK cells), sheep, dog (e.g., MDCK cells from dog kidneys, ATCC CCL34 MDCK (NBL2) or MDCK 33016, deposit number DSM ACC 2219 as described in WO 97/37001); cat, and rodent (e.g., hamster cells such as BHK21-F, HKCC cells, or Chinese hamster ovary (CHO) cells), and may be obtained from a wide variety of developmental stages, including for example, adult, neonatal, fetal, and embryo.

In some embodiments a packaging cell is stably transformed with one or more structural protein expression cassette(s). Structural protein expression cassettes can be introduced into cells using standard recombinant DNA techniques, including transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun” methods, and DEAE- or calcium phosphate-mediated transfection. Structural protein expression cassettes typically are introduced into a host cell as DNA molecules, but can also be introduced as in vitro-transcribed RNA. Each expression cassette can be introduced separately or substantially simultaneously.

An “alphavirus packaging cell” is a cell that contains one or more alphavirus structural protein expression cassettes and that produces recombinant alphavirus particles after introduction of an alphavirus replicon, eukaryotic layered vector initiation system (e.g., U.S. Pat. No. 5,814,482), or recombinant alphavirus particle. In some embodiments, stable alphavirus packaging cell lines are used to produce recombinant alphavirus particles. These are alphavirus-permissive cells comprising DNA cassettes expressing the defective helper RNA or RNAs integrated (preferably stably integrated) into their genomes. See Polo et al., Proc. Natl. Acad. Sci. USA 96, 4598-603, 1999. In some embodiments, the helper RNAs are constitutively expressed but the alphavirus structural proteins are not, because the genes are under the control of an alphavirus subgenomic promoter (Polo et al., 1999). Upon introduction of an alphavirus replicon into the packaging cell by an appropriate method, replicase enzymes are produced and trigger expression of the capsid and glycoprotein genes from the alphavirus subgenomic promoter on the helper RNAs, and output VRPs are produced. Introduction of the replicon can be accomplished by a variety of methods, including both transfection and infection with a seed stock of alphavirus replicon particles. The packaging cell is then incubated under conditions and for a time sufficient to produce packaged alphavirus replicon particles in the culture supernatant.

Thus, packaging cells allow VRPs to act as self-propagating viruses. This technology allows VRPs to be produced in much the same manner, and using the same equipment, as that used for live attenuated vaccines or other viral vectors that have producer cell lines available, such as replication-incompetent adenovirus vectors grown in cells expressing the adenovirus E1A and E1B genes.

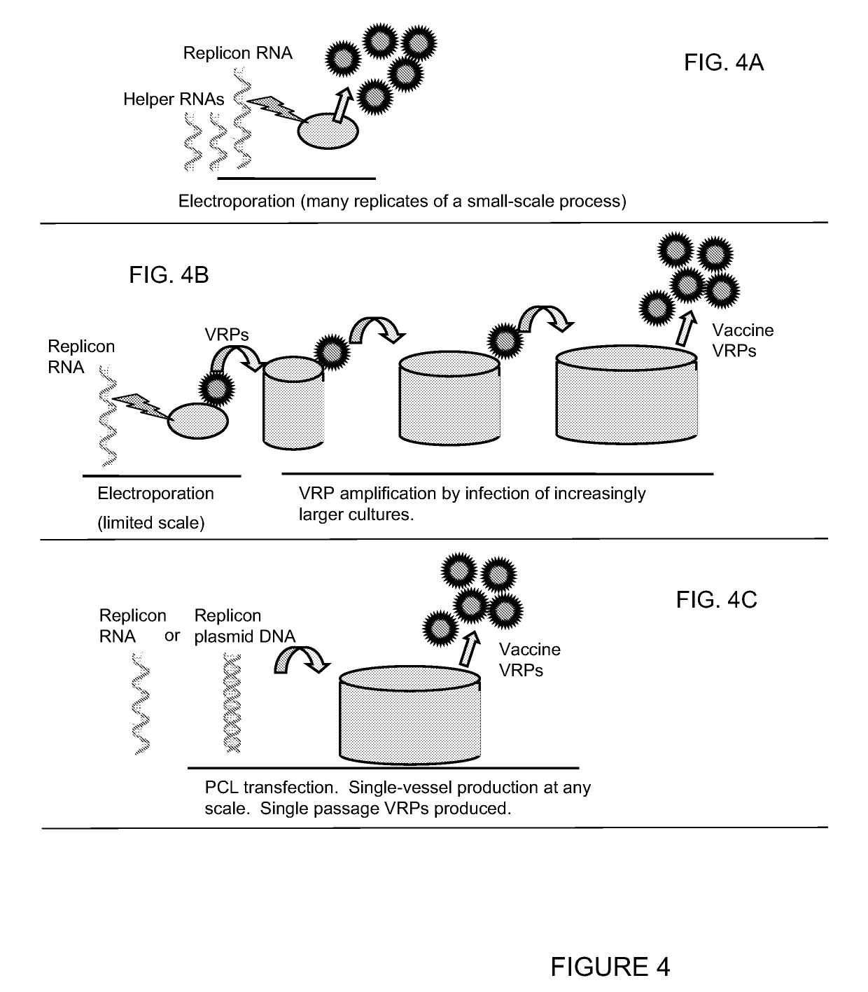

In some embodiments, a two-step process is used: the first step comprises producing a seed stock of virus (e.g., alphavirus) replicon particles by transfecting a packaging cell with a replicon RNA or plasmid DNA-based replicon. A much larger stock of replicon particles is then produced in a second step, by infecting a fresh culture of packaging cells with the seed stock. This infection can be performed using various multiplicities of infection (MOI), including a MOI=0.00001, 0.00005, 0.0001, 0.0005, 0.001, 0.005, 0.01, 0.05, 0.1, 0.5, 1.0, 3, 5, or 10. Preferably infection is performed at a low MOI (e.g., less than 1). Over time, replicon particles can be harvested from packaging cells infected with the seed stock. In some embodiments, replicon particles can then be passaged in yet larger cultures of naive packaging cells by repeated low-multiplicity infection, resulting in commercial scale preparations with the same high titer (for example, see FIG. 4B).

I. VRP Production at Reduced Temperature for Increased VRP Yields

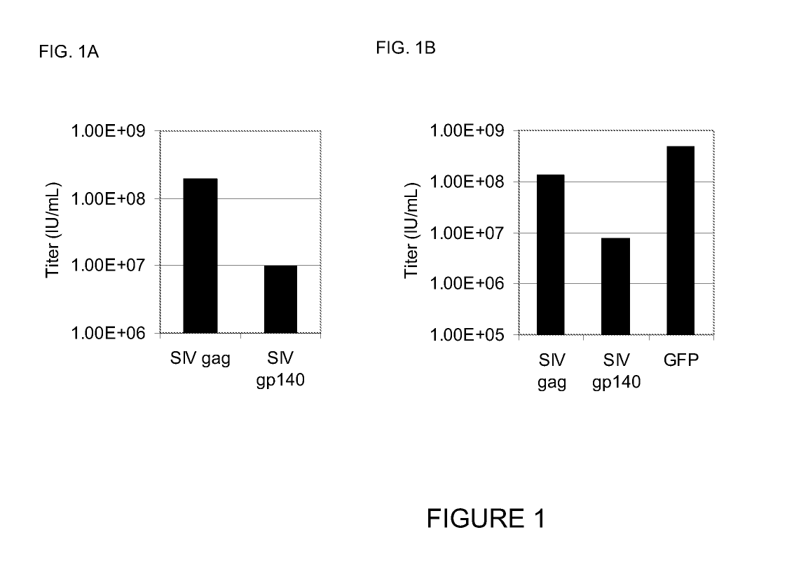

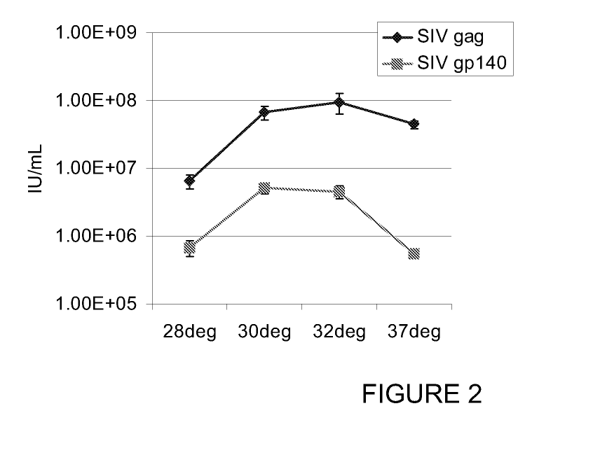

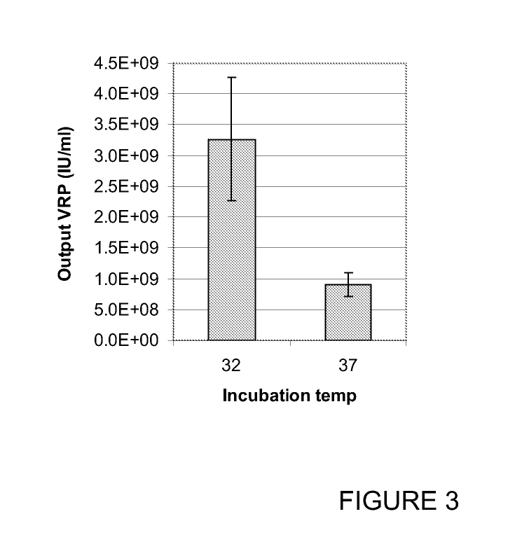

The yield of VRPs from a packaging cell line varies as a function of the encoded antigen. For example, this is true for alphavirus VRPs produced both from BHK cells co-electroporated with replicon and defective helper RNAs and for packaging cells infected with antigen-encoding replicons (FIG. 1). This outcome is markedly improved by lowering the temperature at which infected or transfected packaging cells are incubated. Post-infection or post-transfection incubation of packaging cells typically is carried out at 37° C. Lowering the temperature to a range from about 30° C. and about 35° C.; or about 31° C. and about 34° C.; or about 32° C. and 33° C. (e.g., 30.0° C., 30.5° C., 31° C., 31.5° C., 32° C., 32.5° C., 33° C., 33.5° C., 34° C., 34.5° C., or 35° C.) results in improved VRP yields from packaging cells. For example, reducing the post-infection incubation temperature from 37° C. to 32° C. greatly improves cell viability for packaging cells (FIG. 2). See Examples 2 and 3. Incubation at 32° C. improves VRP yields from packaging cells infected with antigen-encoding VRP by 3 to 10-fold or more for VRPs encoding Simian Immunodeficiency Virus (SIV) gag (encoded by SEQ ID NO:3) or Env (encoded by SEQ ID NO:2) proteins. Reduced temperature also improves VRP yields from packaging cells transfected with replicon RNA (FIG. 3). This is a substantial improvement over previously described VRP production processes.

In some embodiments packaging cells are incubated at the lower temperature from about 18 to about 72 hours (e.g., at least about 18, 20, 24, 26, 28, 30, 32, 36, 40, 48, 60, 65, 70, or 72 hours). In some embodiments packaging cells are incubated at the lower temperature for at least about 18 hours after transfection or infection with the virus (e.g., alphavirus) vector replicon (e.g., at least about 18, 20, 24, 26, 28, 30, 32, 36, 40, 48, 60, 65, 70, or 72 hours). In some embodiments incubation at lower temperature produces replicon particles at a titer at least twice that produced at 37° C.

II. Single-Passage Transfection Strategy for Virus (e.g., Alphavirus) VRP Production

Electroporation has been the most common methodology for alphavirus virus replicon particle (VRP) production because at small scales it is simple and affordable and the only specialized equipment required is a commercially available electroporator. A typical VRP electroporation protocol requires trypsinization of adherent cells, followed by multiple wash steps and electroporation of cells in individual cuvettes, followed by cell plating in adherent format and harvest the next day. However, electroporation may not be cost-effective when performed at industrial scales.

Amplifying VRPs through multiple passages on packaging cells provides time for the encoded antigen to be lost by mutations or deletions in the replicon, and may provide multiple opportunities for replicon RNA to recombine and potentially even form replication-competent virus (RCV). A useful alternative is to introduce replicon RNA to a packaging cell by transfection using electroporation or a nucleic acid delivery reagent (such as lipid:RNA complexes, polyethyleneimine:RNA complexes, RNA-containing liposomes, etc.). Transfection of cells with these reagents is scalable and can be performed in any size culture vessel, for either suspension or adherent cells. With transfection of packaging cells, VRP production can be limited to a single passage, regardless of the production scale, in which replicon RNA is transfected and the VRPs are harvested for downstream processing within a few days of transfection. Because cells that receive the replicon by transfection will rapidly produce VRPs which infect neighboring cells, even a fairly low efficiency transfection would initiate a spreading infection that yields high titers of VRPs. This approach is simpler and more scalable than electroporation-based systems, and eliminates the need for repeated passages previously used for VRP production by packaging cells (FIG. 4).

III. VRP Production by DNA Replicon Plasmid Transfection into Packaging Cell Lines Using Nucleic Acid Delivery Reagents

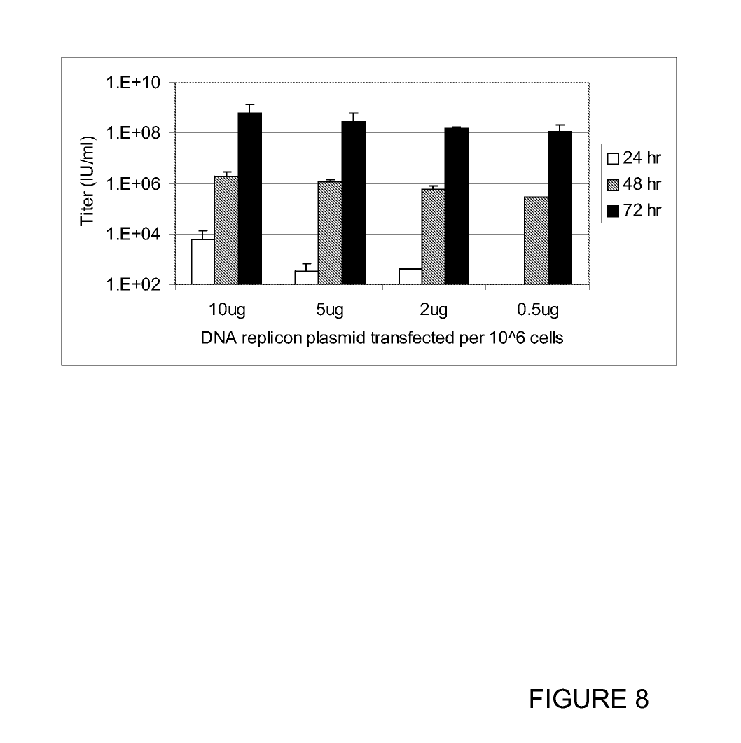

In some embodiments replicon-expressing DNA is used instead of replicon RNA in the transfection step. In this case, a plasmid is used in which transcription of a replicon RNA is placed under the control of a eukaryotic promoter, such as the CMV immediate-early promoter. Transfection of this plasmid into a packaging cell results in replicon production in the cell nucleus, followed by replicon RNA transport to the cytoplasm, translation of the replicon nonstructural proteins and the onset of RNA replication, output VRP production, spreading infection in the packaging cell culture, and ultimately high titers of VRP for harvest (FIG. 8). See Example 7. This DNA transfection approach eliminates the in vitro production of transcribed replicon RNA in order to make VRPs, simplifies VRP production process, and reduces cost.

In some embodiments a DNA replicon plasmid comprises a DNA-dependent RNA polymerase promoter driving the transcription of an RNA cassette which comprises an alphavirus 5′ RNA replication signal, an open reading frame encoding alphavirus nonstructural proteins, one or more cassettes which direct expression of a heterologous gene(s) from the alphavirus replicon RNA, an alphavirus 3′ untranslated region (UTR) including RNA replication signal(s), a polyA tract of 10-30 nucleotides, a hepatitis delta virus ribozyme sequence, and a transcriptional termination sequence.

Certain non-limiting embodiments are set forth below.

1. A method of producing alphavirus replicon particles, comprising culturing a packaging cell under conditions suitable for production of alphavirus replicon particles, wherein the conditions comprise a temperature in a range from about 30° C. to about 35° C., wherein the packaging cell comprises:
(a) one or more alphavirus structural protein expression cassettes directing expression of alphavirus structural proteins; and
(b) a vector selected from the group consisting of an alphavirus vector construct, an RNA vector replicon, a DNA replicon plasmid, a eukaryotic layered vector initiation system, and an alphavirus vector particle,
whereby alphavirus vector particles are produced.
2. The method of embodiment 1 wherein the packaging cell comprises (i) a first alphavirus structural protein expression cassette which directs expression of an alphavirus capsid protein; and (ii) a second alphavirus structural protein expression cassette which directs expression of at least one of an alphavirus E1 glycoprotein and an alphavirus E2 glycoprotein.
3. The method of embodiment 1 further comprising the step of introducing the vector into the packaging cell.
4. The method of embodiment 3 wherein the vector is introduced into the packaging cell by infection.
5. The method of embodiment 3 wherein the vector is introduced into the packaging cell by transfection.
6. The method of embodiment 5 wherein the vector is introduced into the packaging cell by electroporation.
7. The method of embodiment 3 wherein the vector is the DNA replicon plasmid.
8. The method of embodiment 7 wherein the conditions comprise a temperature of about 32° C.
9. The method of embodiment 5 wherein the packaging cell is transfected using a transfection reagent selected from the group consisting of a lipoplex, calcium phosphate, a liposome, polyethyleneimine (PEI), a cationic nanoemulsion, a lipid nanoparticle, protamine, polyarginine, polylysine, and a cationic lipid.
10. The method of embodiment 9 wherein the transfection reagent is a lipoplex and the lipoplex comprises 1:1 (w/w) 1,2-dioleoyl-3-trimethylammonium-propane (chloride salt) (DOTAP): 1,2-dioleoyl-sn-glycero-3-phosphoethanolamine (DOPE).
11. The method of embodiment 9 wherein the transfection reagent is PEI.
12. The method of embodiment 1 wherein the conditions comprise a temperature of about 32° C.
13. The method of embodiment 1 wherein the packaging cell is derived from an avian cell.
14. The method of embodiment 15 wherein the avian cell is a duck cell.
15. A method of producing alphavirus replicon particles, comprising:

(a) introducing into a packaging cell by transfection a vector selected from the group consisting of an alphavirus vector construct, an RNA vector replicon, a DNA replicon plasmid, a eukaryotic layered vector initiation system, and an alphavirus vector particle, wherein the packaging cell comprises one or more alphavirus structural protein expression cassettes directing expression of alphavirus structural proteins; and

(b) culturing the packaging cell under conditions suitable for production of alphavirus replicon particles,

whereby alphavirus vector particles are produced.
16. The method of embodiment 15 wherein the packaging cell comprises (i) a first alphavirus structural protein expression cassette which directs expression of an alphavirus capsid protein; and (ii) a second alphavirus structural protein expression cassette which directs expression of at least one of an alphavirus E1 glycoprotein and an alphavirus E2 glycoprotein but not the alphavirus capsid protein
17. The method of embodiment 15 wherein the packaging cell is transfected using electroporation.
18. The method of embodiment 15 wherein the packaging cell is transfected using a transfection reagent selected from the group consisting of a lipoplex, calcium phosphate, a liposome, polyethyleneimine (PEI), a cationic nanoemulsion, a lipid nanoparticle, protamine, polyarginine, polylysine, and a cationic lipid.
19. The method of embodiment 18 wherein the transfection reagent is a lipoplex and the lipoplex comprises 1:1 (w/w) 1,2-dioleoyl-3-trimethylammonium-propane (chloride salt) (DOTAP): 1,2-dioleoyl-sn-glycero-3-phosphoethanolamine (DOPE).
20. The method of embodiment 18 wherein the transfection reagent is PEI.
21. The method of embodiment 15 wherein the conditions comprise a temperature range of about 30° C. and about 35° C.
22. The method of embodiment 21 wherein the conditions comprise a temperature of about 32° C.
23. The method of embodiment 15 wherein the packaging cell is derived from an avian cell.
24. The method of embodiment 23 wherein the avian cell is a duck cell.
25. The method of embodiment 15 wherein the vector is the DNA replicon plasmid.
26. The method of embodiment 25 wherein the conditions comprise a temperature of about 32° C.
27. A method of producing alphavirus replicon particles, comprising a step of culturing a packaging cell under conditions suitable for production of alphavirus replicon particles, wherein the conditions comprise a temperature range from about 30° C. and about 35° C., whereby alphavirus replicon particles are produced.
28. The method of embodiment 27, comprising the steps:
(a) introducing an alphavirus vector into a packaging cell; and
(b) culturing the packaging cell under conditions suitable for production of alphavirus replicon particles, wherein the conditions comprise a temperature range from about 30° C. and about 35° C.
29. The method of embodiment 28, wherein the vector is introduced into the packaging cell by infection.
30. The method of embodiment 28, wherein the vector is introduced into the packaging cell by transfection.
31. The method of embodiment 30, wherein the vector is introduced into the packaging cell by electroporation.
32. The method of any of embodiments 28-31, wherein the alphavirus vector is a self-replicating alphavirus-derived RNA molecule.
33. The method of embodiment 32, wherein the vector is introduced into the packaging cell by infection or transfection of the cell with the self-replicating alphavirus-derived RNA molecule.
34. The method of embodiment 32, wherein the vector is introduced into the packaging cell by infection or transfection of the cell with a DNA molecule encoding the self-replicating alphavirus-derived RNA molecule.
35. The method of any preceding embodiment, wherein the conditions comprise a temperature range from about 31° C. and about 34° C.
36. The method of embodiment 35, wherein the conditions comprise a temperature of about 32° C.
37. The method of any preceding embodiment, wherein the packaging cells are cultured at said temperature for at least 6 hours after introduction of an alphavirus vector to the packaging cells.
38. The method of embodiment 37, wherein the packaging cells are cultured at said temperature for at least 12 hours after introduction of an alphavirus vector to the packaging cells.
39. The method of embodiment 38, wherein the packaging cells are cultured at said temperature for at least 24 hours after introduction of an alphavirus vector to the packaging cells.
40. The method of any of embodiments 27-39, wherein the packaging cell contains one or more structural protein expression cassettes which encode an alphavirus capsid protein and alphavirus E1 and E2 envelope glycoproteins.
41. The method of embodiment 40, wherein said one or more structural protein expression cassettes comprise one or more alphavirus-derived replication-competent RNA helper vectors which encode an alphavirus capsid protein and alphavirus E1 and E2 envelope glycoproteins.
42. The method of embodiment 40, wherein said one or more structural protein expression cassettes comprise one or more DNA molecules encoding one or more alphavirus-derived replication-competent RNA helper vectors which encode an alphavirus capsid protein and alphavirus E1 and E2 envelope glycoproteins.
43. The method of embodiment 42, wherein said one or more structural protein expression cassettes are stably integrated in the packaging cell.
44. The method of any of preceding embodiment, wherein the packaging cell contains:
(i) a first alphavirus structural protein expression cassette which directs expression of an alphavirus capsid protein; and
(ii) a second alphavirus structural protein expression cassette which directs expression of at least one of an alphavirus E1 glycoprotein and an alphavirus E2 glycoprotein.
45. The method of any preceding embodiment, wherein the packaging cell is derived from a mammalian cell.
46. The method of embodiment 45, wherein the packaging cell is derived from an avian cell.
47. The method of embodiment 46, wherein the packaging cell is derived from a duck cell.
48. The method of embodiment 30 wherein the packaging cell is transfected using a transfection reagent selected from the group consisting of a lipoplex, calcium phosphate, a liposome, polyethyleneimine (PEI), a cationic nanoemulsion, a lipid nanoparticle, protamine, polyarginine, polylysine, and a cationic lipid.
49. The method of embodiment 48 wherein the transfection reagent is a lipoplex and the lipoplex comprises 1:1 (w/w) 1,2-dioleoyl-3-trimethylammonium-propane (chloride salt) (DOTAP): 1,2-dioleoyl-sn-glycero-3-phosphoethanolamine (DOPE).
50. The method of embodiment 48 wherein the transfection reagent is PEI.
51. The method of any preceding embodiment, wherein at least 1×10⁶ IU/ml, at least 1×10⁷ IU/ml, or at least 1×10⁸ IU/ml, of alphavirus replicon particles are produced.
52. A method of producing alphavirus replicon particles, comprising:
(a) introducing into a packaging cell by transfection an alphavirus vector;
(b) culturing the packaging cell under conditions suitable for production of alphavirus replicon particles,
whereby alphavirus vector particles are produced.
53. The method of embodiment 52, wherein the packaging cell is transfected using a transfection reagent selected from the group consisting of a lipoplex, calcium phosphate, a liposome, polyethyleneimine (PEI), a cationic nanoemulsion, a lipid nanoparticle, protamine, polyarginine, polylysine, and a cationic lipid.
54. The method of embodiment 53, wherein the transfection reagent is a lipoplex and the lipoplex comprises 1:1 (w/w) 1,2-dioleoyl-3-trimethylammonium-propane (chloride salt) (DOTAP): 1,2-dioleoyl-sn-glycero-3-phosphoethanolamine (DOPE).
55. The method of embodiment 53, wherein the transfection reagent is PEI.
56. The method of any of embodiments 52-55, wherein the alphavirus vector is a self-replicating alphavirus-derived RNA molecule.
57. The method of embodiment 56, wherein the vector is introduced into the packaging cell by transfection of the cell with the self-replicating alphavirus-derived RNA molecule.
58. The method of embodiment 56, wherein the vector is introduced into the packaging cell by transfection of the cell with a DNA molecule encoding the self-replicating alphavirus-derived RNA molecule.
59. The method of any of embodiments 52-58, which comprises culturing the packaging cells at a temperature range from about 30° C. and about 35° C.
60. The method of embodiment 59, wherein the conditions comprise a temperature range from about 31° C. and about 34° C.
61. The method of embodiment 60, wherein the conditions comprise a temperature of about 32° C.
62. The method of any of embodiments 52-61, wherein the packaging cell contains one or more structural protein expression cassettes which encode an alphavirus capsid protein and alphavirus E1 and E2 envelope glycoproteins.
63. The method of embodiment 62, wherein said one or more structural protein expression cassettes comprise one or more alphavirus-derived replication-competent RNA helper vectors which encode an alphavirus capsid protein and alphavirus E1 and E2 envelope glycoproteins.
64. The method of embodiment 363, wherein said one or more structural protein expression cassettes comprise one or more DNA molecules encoding one or more alphavirus-derived replication-competent RNA helper vectors which encode an alphavirus capsid protein and alphavirus E1 and E2 envelope glycoproteins.
65. The method of embodiment 64, wherein said one or more structural protein expression cassettes are stably integrated in the packaging cell.
66. The method of any of embodiments 52-65, wherein the packaging cell contains:
(i) a first alphavirus structural protein expression cassette which directs expression of an alphavirus capsid protein; and
(ii) a second alphavirus structural protein expression cassette which directs expression of at least one of an alphavirus E1 glycoprotein and an alphavirus E2 glycoprotein.
67. The method of any of embodiments 52-66, wherein the packaging cell is derived from a mammalian cell.
68. The method of embodiment 67, wherein the packaging cell is derived from an avian cell.
69. The method of embodiment 68, wherein the packaging cell is derived from a duck cell.
70. The method of any of embodiments 52-69, wherein at least 1×10⁶ IU/ml, at least 1×10⁷ IU/ml, or at least 1×10⁸ IU/ml, of alphavirus replicon particles are produced.
71. The method of any preceding embodiment, wherein the number of alphavirus replicon particles produced in the method is at least 2-fold, at least 5-fold, or at least 10-fold, the number obtainable by performing a corresponding method in which the packaging cells are cultured at a temperature above about 35° C.
72. The method of any of embodiments 1-71, wherein the alphavirus vector comprises a heterologous coding sequence encoding an antigen derived from a pathogen.
73. The method of any of embodiments 1-71, wherein the alphavirus vector comprises a heterologous coding sequence encoding a glycoprotein.
74. The method of any preceding embodiment, wherein the alphavirus replicon particle comprises a Venezuelan Equine Encephalitis (VEE) derived vector construct packaged with Sindbis (SIN) capsid and/or envelope glycoproteins.
75. The method according to any preceding embodiment, which further comprises one or more steps for separating the alphavirus vector replicons from the packaging cells, whereby a composition substantially free of packaging cells and containing the alphavirus vector replicons is produced.
76. The method according to any preceding embodiment, which further comprises one or more steps for purifying the alphavirus vector replicons from the culture medium, whereby a purified composition containing the alphavirus vector replicons is produced.
77. The method according to any preceding embodiment, which further comprises one or more steps for formulating the alphavirus vector replicons into a pharmaceutical composition, whereby a pharmaceutical composition containing the alphavirus vector replicons is produced.
78. The method according to any preceding embodiment, wherein said packaging cells are transfected or infected with said vector in suspension culture.
79. The method according to any preceding embodiment, wherein said packaging cells are transfected or infected with said vector in adherent culture.
80. The method according to any preceding embodiment, wherein said packaging cells are cultured at said temperature in suspension culture.
81. The method according to any preceding embodiment, wherein said packaging cells are cultured at said temperature in adherent culture.

All patents, patent applications, and references cited in this disclosure are expressly incorporated herein by reference. The above disclosure is a general description. A more complete understanding can be obtained by reference to the following specific examples, which are provided for purposes of illustration only.

### Example 1

### Materials and Methods Used in the Examples

BHK-V cells were cultured in DMEM+5% fetal bovine serum+penicillin, streptomycin, and 2 mM L-glutamine.

Packaging cell line derivation was performed by stable transfection of AGE.CR cells (Jordan et al., Vaccine 27: 748-56, 2009) with one or two DNA plasmids encoding the two defective helper RNAs, similar to previously published work in BHK cells (Polo et al., 1999). In this case, one defective helper encoded a neomycin resistance gene followed by the Sindbis glycoprotein genes under the control of an alphavirus subgenomic promoter, and the second defective helper encoded only the Sindbis capsid gene under the control of an alphavirus subgenomic promoter. Adherent packaging cell lines were cultured in tissue culture-treated flasks in DMEM/F12+5% fetal bovine serum+penicillin, streptomycin, and 2 mM L-glutamine at 37 C in 7.5% CO₂; suspension packaging cells were cultured in Adenovirus Expression Medium (AEM, Invitrogen) with penicillin, streptomycin, and 4 mM L-glutamine on a shaking platform at 37 C in 7.5% CO₂ at 100 rpm.

Replicon plasmids for in vitro transcription were constructed as previously described (Perri et al., J Virol 77, 10394-403, 2003). For cloning various genes of interest into the replicon plasmid, appropriate restriction sites were added to the 5′ and 3′ ends of the gene of interest cassettes by PCR, followed by restriction digestion of the replicon plasmid and insert, with subsequent ligation, colony selection, and sequencing.

Electroporation was performed as previously described (Perri et al., 2003). For VRP production by packaging cell infection, seed VRPs were produced from electroporated BHK-V cells. Adherent packaging cells were inoculated with VRPs in DMEM/F12+5% fetal bovine serum+penicillin, streptomycin, and 2 mM L-glutamine, and supernatant samples collected at 24-72 hours post-infection for determination of output VRP titers.

VRP Production by Adherent Packaging Cell Transfection with Replicon RNA.

Adherent packaging cells were placed in DMEM/F12+1% fetal bovine serum+2 mM L-glutamine with 2 μg RNA transfection complex per 10⁶ cells for 4 hours. Transfection medium was removed by aspiration, and replaced with DMEM/F12+5% fetal bovine serum+2 mM L-glutamine, and supernatant samples collected 24-72 hours post-transfection for determination of output VRP titers.

Liposome Preparation.

DOTAP (1,2-Dioleoyl-3-Trimethylammonium-Propane [Chloride Salt], Avanti Polar Lipids, Alabaster, Ala.) and DOPE (1,2-Dioleoyl-sn-Glycero-3-Phosphoethanolamine, Avanti Polar Lipids, Alabaster, Ala.) were dissolved in chloroform at 10 mg/ml. 0.5 ml aliquots of DOTAP and DOPE in chloroform were placed into 3 ml glass vials and lipid films were prepared by evaporation of the chloroform using a rotary evaporator (Buchi model number R200) under reduced pressure (300 milliTorr, 30 min, water bath 50° C.). Residual chloroform was then removed by placing the samples overnight in a Labconco freeze dryer under reduced pressure. The lipid film was then hydrated as an multilamellar vesicle (MLV) by the addition of 1.0 mL of DEPC treated water (EMD Biosciences, San Diego, Calif.), high speed vortexing on a bench top vortexer, and incubation at 50° C. in a heating block for 10 minutes, followed by additional high speed vortexing on a bench top vortexer. After lipid reconstitution, lipoplexes were made by mixing with RNA or DNA. Each μg of nucleotide was assumed to contain 3 nmoles of anionic phosphate, each μg of DOTAP was assumed to contains 0.14 nmoles of cationic nitrogen. Complexes with RNA or DNA were formulated by diluting liposomes to 1.675 mg/ml in RNAse-free water (for 4:1 N:P ratio in lipoplex), then mixing 1:1 by volume with replicon RNA or DNA at 0.1 μg/μl in RNAse-free water and allowing 30 minutes at 4° C. for complexation. The nucleotide solution was always added to the liposome solution. Final lipoplexes had a 4:1 Nitrogen:Phosphate ratio and an RNA or DNA concentration of 0.05 μg per μl.

Determination of infectious VRP titers was performed in 96-well plate format. 50,000

BHK-V cells per well were plated in growth medium and allowed to adhere for 4 hours at 37 C. Serial 10-fold dilutions of VRP samples were made in a separate plate, then plating medium was removed from all BHK wells and replaced with 100 ul of serial VRP dilutions. VRP titers were determined by counting fluorescent cells (for GFP-expressing replicons) or by immunostaining for the VEE nonstructural proteins and counting stained cells.

### Example 2

### Reduced Temperature Improves VRP Yields from Infected Packaging Cells

Packaging cells were derived from AGE.CR cells as described in Example 1. These packaging cells were infected with VRP encoding SIV gag or SIV gp140 and incubated at 28° C., 30° C., 32° C., or 37° C. for 48 hours. Culture supernatant was then harvested and the VRP titer determined by limiting dilution titration assay as described above.

This example demonstrates that reduced temperature improves yield of VRPs encoding vaccine-relevant antigens from packaging cells into which VRP were introduced by infection.

### Example 3

### Reduced Temperature Improves VRP Yields in Transfection-Based VRP Production from Packaging Cells

Adherent packaging cells were transfected with 2 μg GFP replicon RNA per 10⁶ cells using DOTAP:DOPE transfection reagent as described in Example 4, incubated for 48 hours at 32° C. or 37° C. Culture supernatant was then harvested, and the VRP titer was determined by limiting dilution titration assay as described above.

This example demonstrates that reduced temperature improves yield of VRPs from packaging cells into which replicon RNA was introduced by transfection.

### Example 4

### Transfection of Packaging Cells Using DOTAP:DOPE Lipoplexes

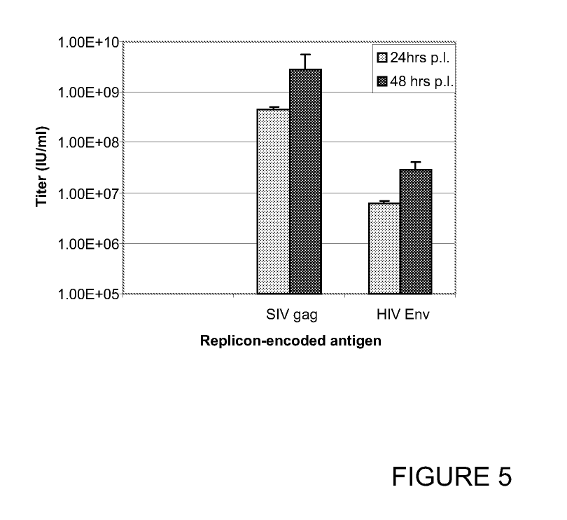

Replicon RNA was introduced into adherent AGE.CR pIX clone packaging cells using cationic lipid-mediated RNA delivery with lipoplexes of 1:1 (w/w) 1,2-dioleoyl-3-trimethylammonium-propane (chloride salt) (DOTAP):1,2-dioleoyl-sn-glycero-3-phosphoethanolamine (DOPE) prepared as described in Example 1. Transfection of packaging cells with replicon RNA by this method results in rapid and high-yield VRP production; at 24 to 48 hours post-transfection, yields were in excess of 10⁹ IU/ml. Furthermore, yields of 1×10⁸ to over 10⁹ IU/ml were achieved with replicons expressing relevant vaccine antigens including HIV env protein and SIV gag protein (FIG. 5). This data shows that a scalable, single-passage VRP production strategy based upon transfection of replicon RNA into packaging cell is feasible for producing VRPs encoding a variety of antigens.

### Example 5

### Transfection of Packaging Cells Using PEI

Replicon RNA was introduced into adherent AGE.CR pIX clone packaging cells using a polyplex preparation. Branched 50 kd, or linear 25 kd molecular weight polyethyleneimine (PEI) (Polysciences, Inc. Warrington, Pa.) was added to RNase free water (Ambion, Austin, Tex.) at 2 mg/ml. Each solution was brought to 80° C. until the PEI was fully dissolved. The resulting stock solution was adjusted to pH 7.2 with 2M HCl. The stock solution was then diluted in 100 mM citrate buffer pH 6.0 (Teknova, Hollister, Calif.) to create a working stock at a concentration of 1 mg/ml. RNA complexes were prepared by diluting 7.5 μg RNA in a total of 750 RNase free water. The RNA solution was added to a PEI solution containing either 4.850 or 9.700 of the PEI working stock solution diluted in a total of 750 RNase free water (5:1 N/P or 10:1 N/P ratio respectively). The resulting mixtures were allowed to sit at 4° C. for 30 minutes to allow for complexation.

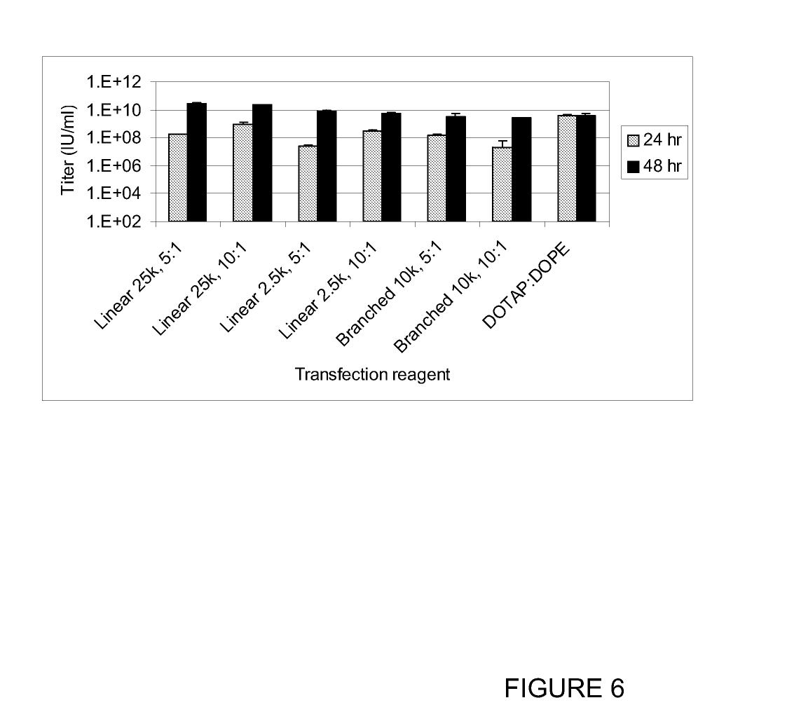

The yield of VRPs using PEI-mediated transfection as described above was similar to the yield obtained using DOTAP:DOPE lipoplexes (FIG. 6).

### Example 6

### Transfection-Based VRP Production in Suspension

While the VRP yields produced by transfection of adherent cells were very high, suspension cells are a more scalable and cost-effective cell culture format for industrialization. We demonstrated the feasibility of transfection-based VRP production in suspension and by replicon RNA transfection into suspension AGE.CR packaging cell using DOTAP:DOPE as the transfection reagent.

For VRP production by suspension packaging cell transfection with replicon RNA or DNA, suspension packaging cells were transfected at 10⁶ viable cells per ml in AEM+4 mM L-glutamine with 2 μg RNA or DNA transfection complex per ml for 4 hours at 100 rpm. Cells were pelleted gently for 5 mins at 750 rpm, then transfection medium was removed by aspiration, and replaced with DMEM/F12+5% fetal bovine serum+2 mM L-glutamine. Cells were returned to shaking and supernatant samples were collected 24-72 hours post-transfection for determination of output VRP titers.

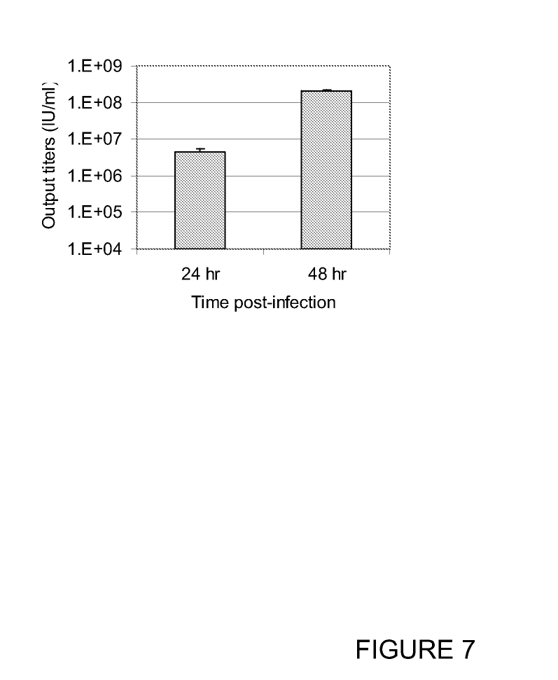

Suspension packaging cell transfection with GFP-encoding replicon RNA resulted in high VRP yields (10⁹ IU/ml) (FIG. 7). This data confirms that transfection of packaging cell with replicon RNA can be performed using different transfection reagents and cell culture formats.

### Example 7

### VRP Production by DNA Replicon Plasmid-Transfected Packaging Cells

DNA-launched replicon plasmids were generated by ligation of DNA fragments containing, in 5′-3′ order, the following: the CMV immediate-early promoter, the VEE 5′ untranslated region, the VEE nonstructural proteins with inserted Sindbis packaging signal, the VEE subgenomic promoter, a cloning site for insertion of genes of interest, the SIN3′-UTR, a polyA tail, Hepatitis Delta Virus ribozyme sequences, the BGH polyA-signal, the kanamycin resistance gene, and the colE1 origin of replication. All VEE sequences were derived from the Trinidad Donkey strain of VEE. Genes of interest were cloned into the resulting plasmid as described above.

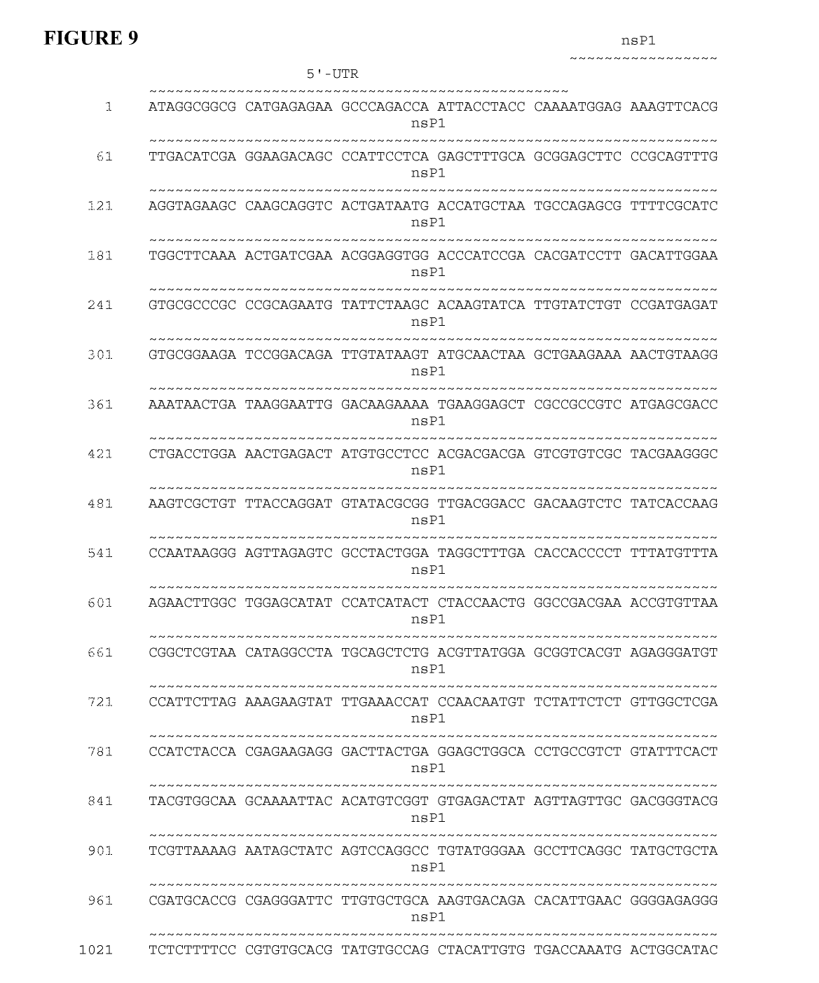

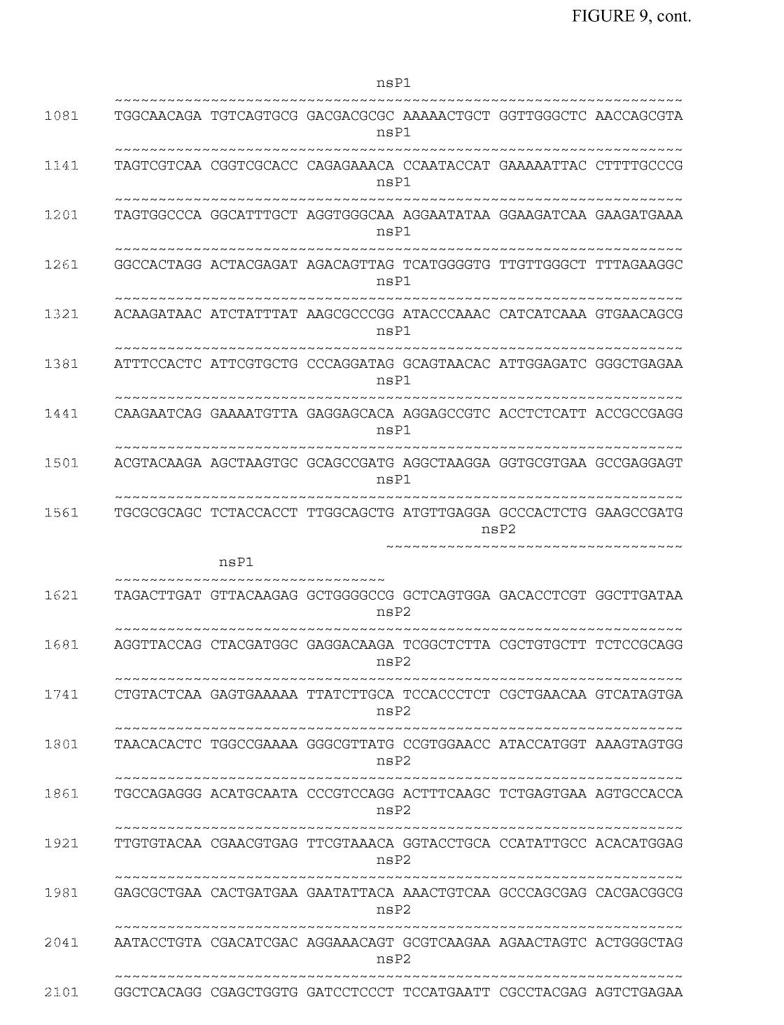

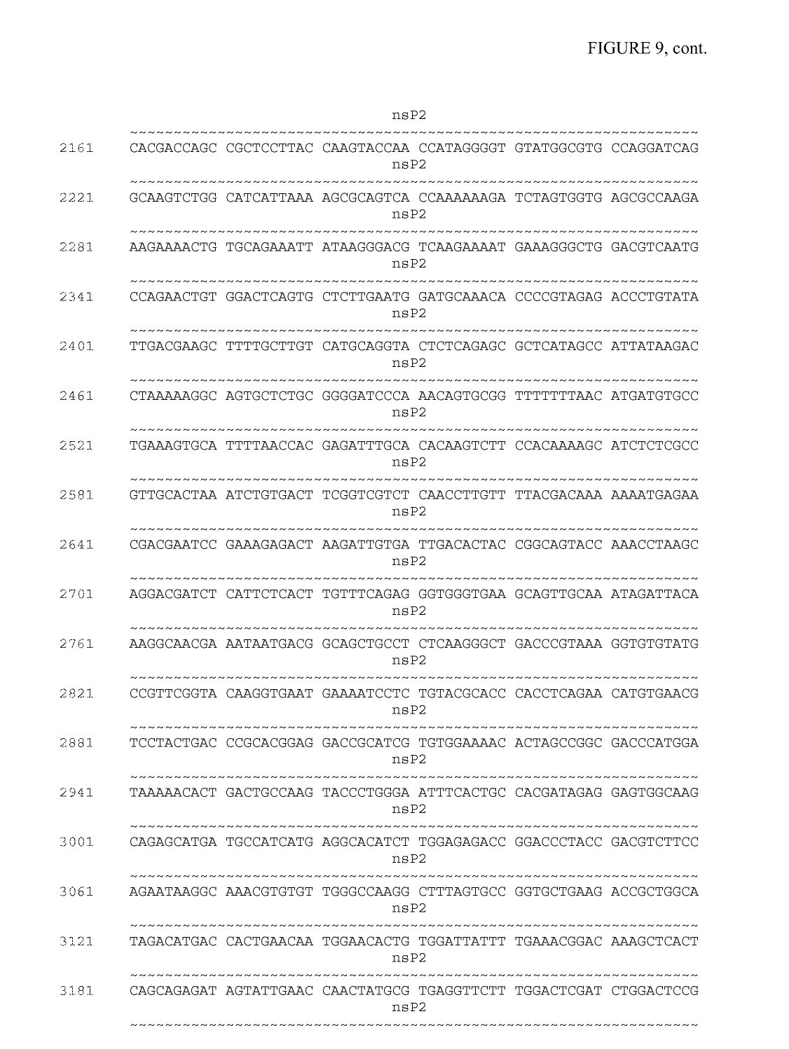

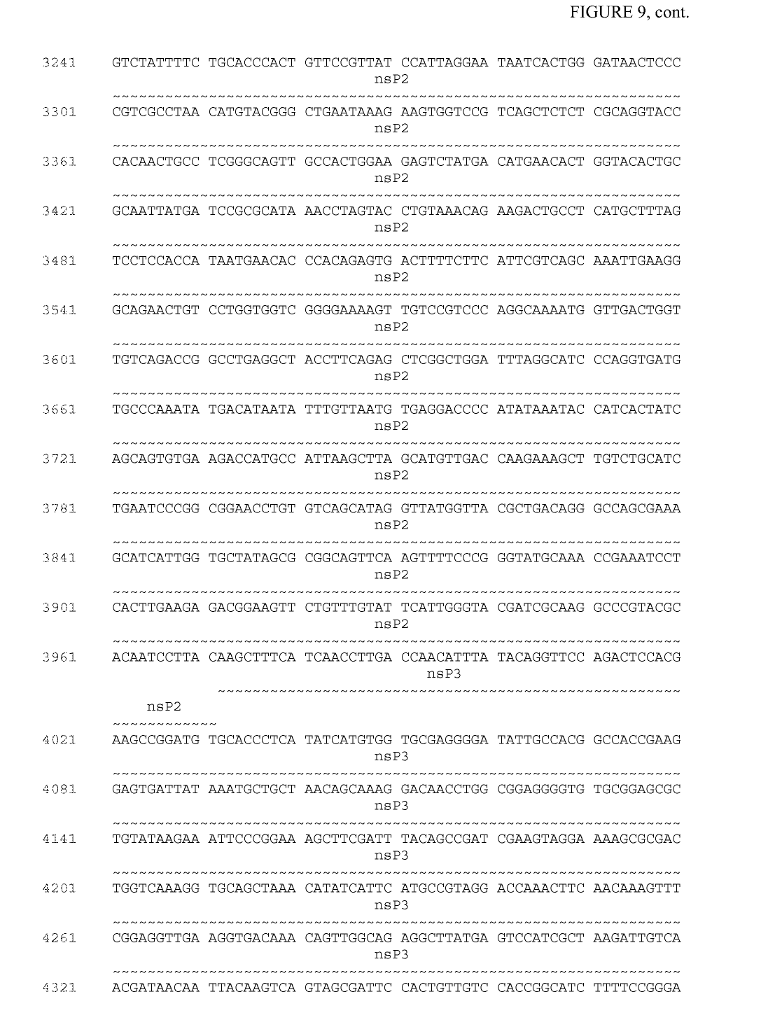

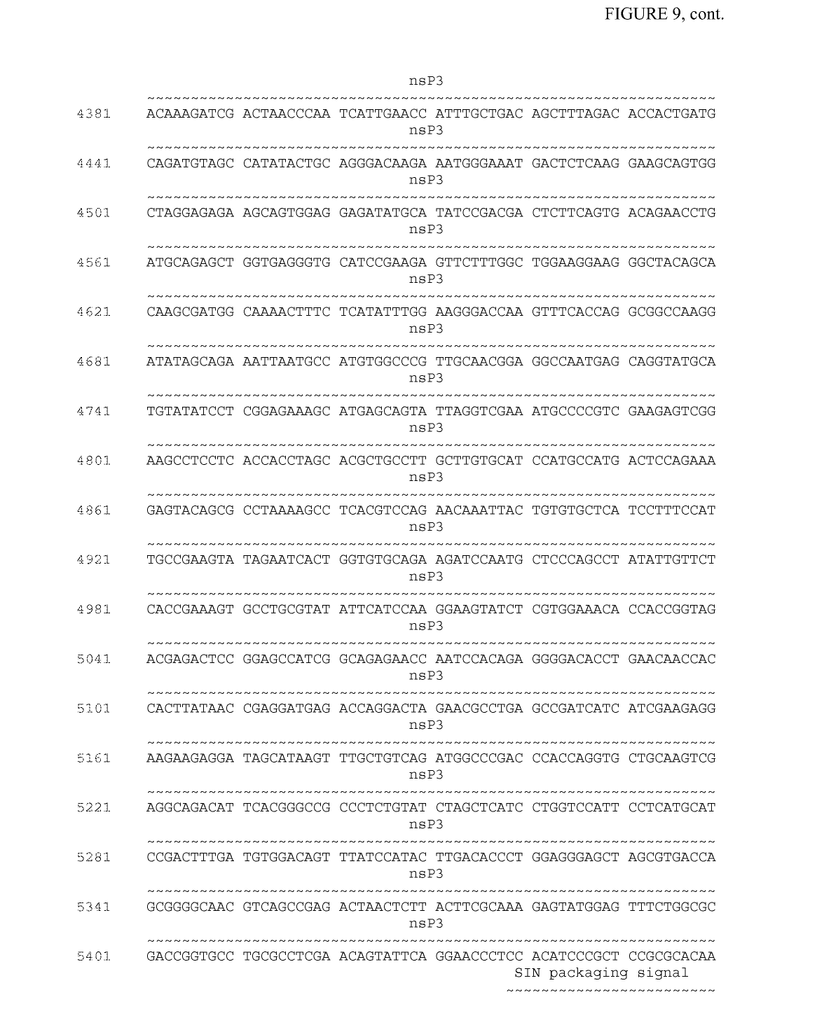

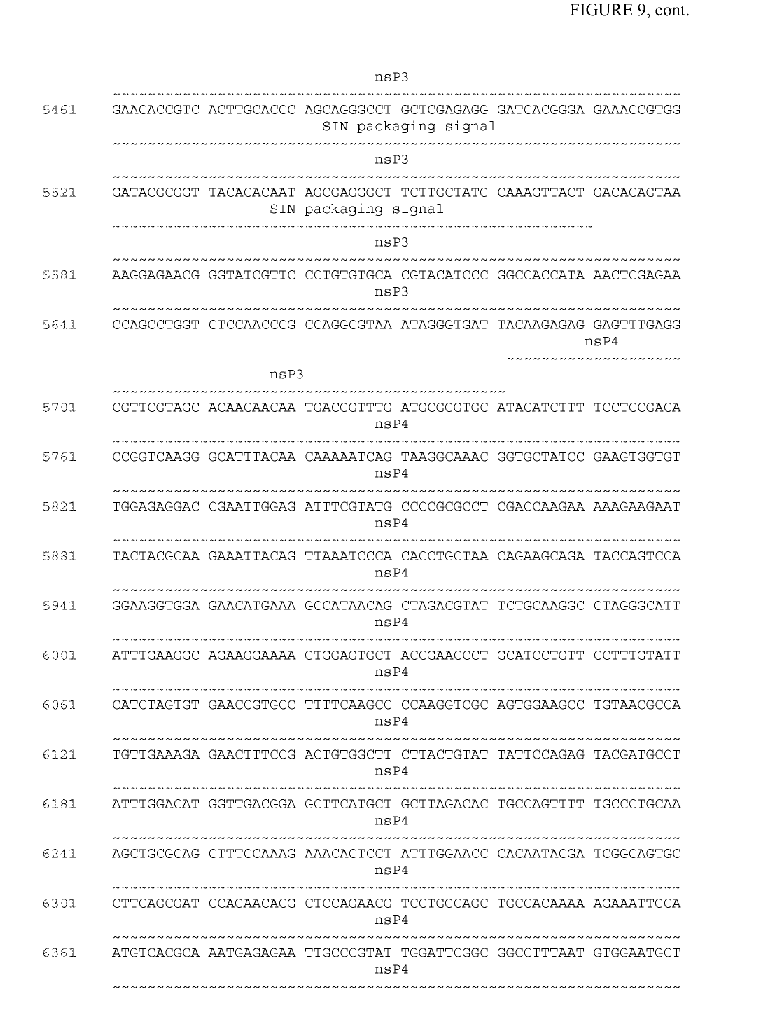

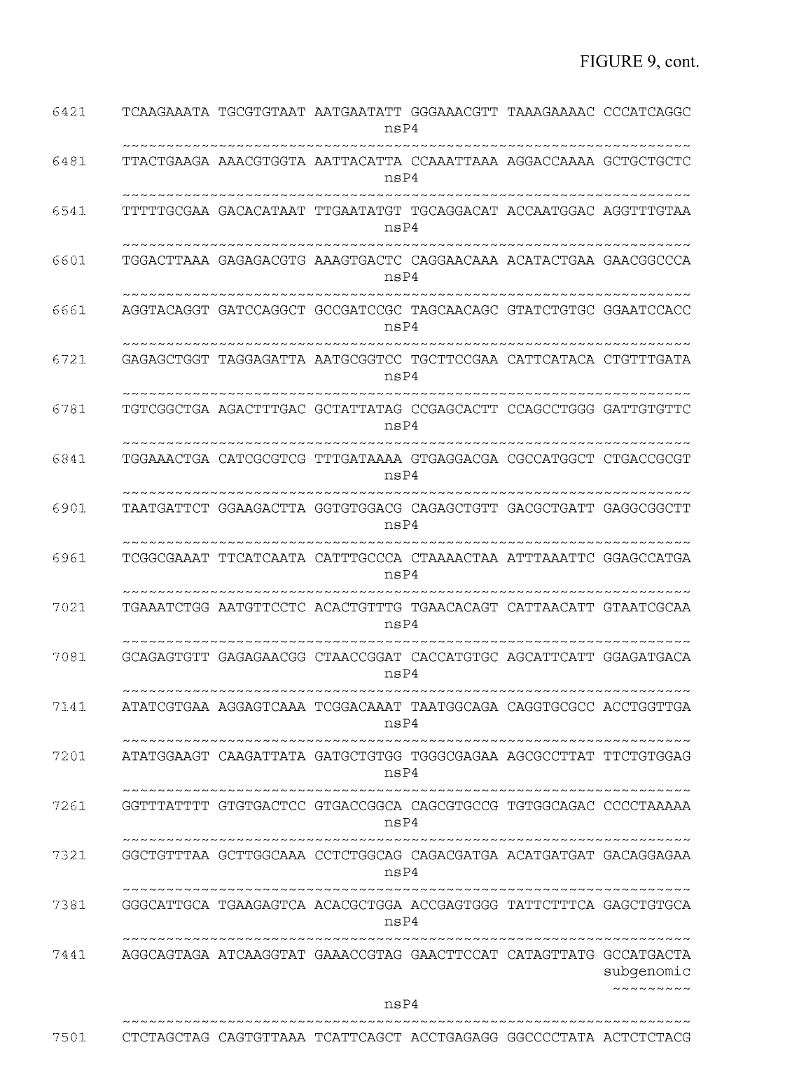

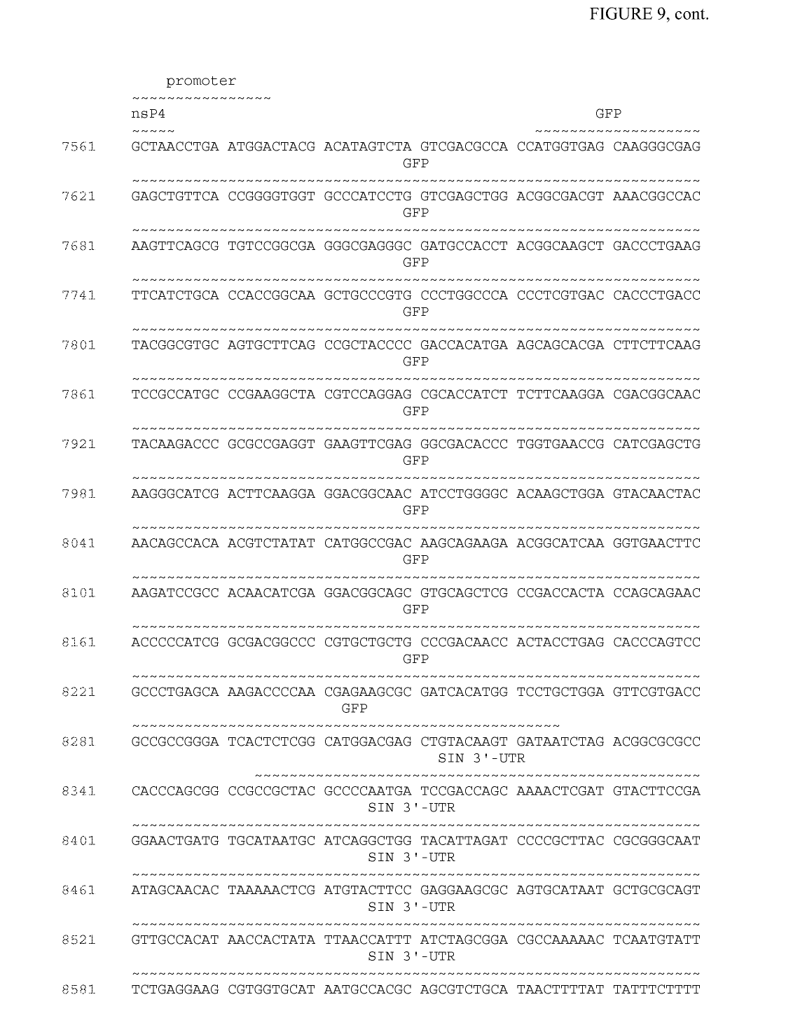

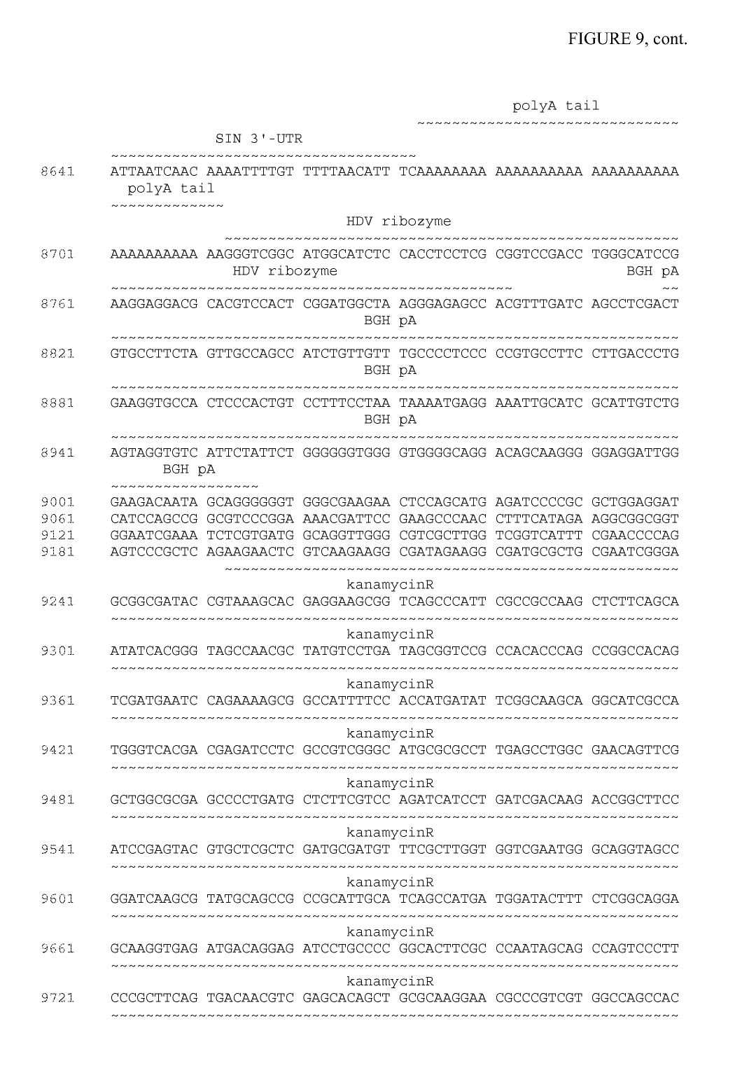

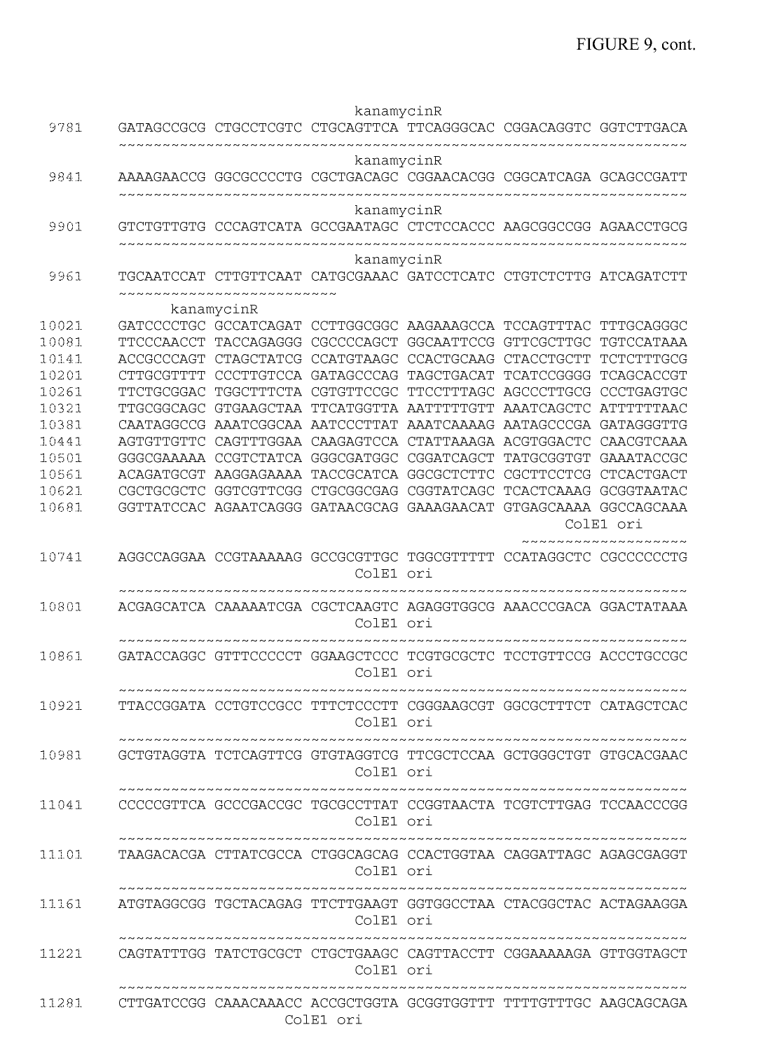

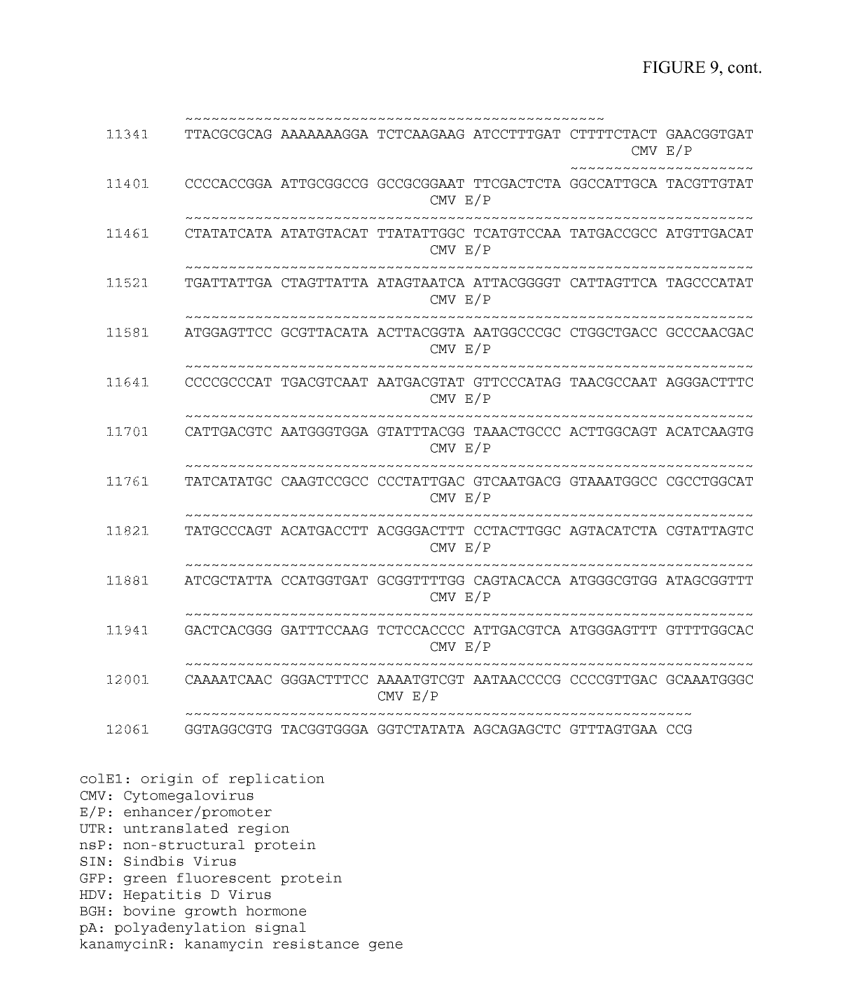

We constructed a DNA plasmid driving the expression of a VEE/SIN chimeric replicon encoding GFP (FIG. 9; SEQ ID NO:1). Transfection of this plasmid into suspension packaging cell using DOTAP:DOPE resulted in VRP yields of >10⁸ IU/ml by 72 hours post-transfection at a variety of plasmid doses, demonstrating the feasibility of this approach for VRP production (FIG. 8). As is the case with RNA replicon transfection, DNA replicon plasmid transfection may be improved for industrialization by using alternative transfection reagents.

1. A method of producing virus replicon particles, comprising a step of culturing a packaging cell under conditions suitable for production of virus replicon particles, wherein the conditions comprise a temperature in a range from about 30° C. and about 35° C., whereby virus replicon particles are produced.

2. The method of claim 1, wherein the packaging cell comprises:
(a) one or more virus structural protein expression cassettes directing expression of virus structural proteins; and
(b) a virus vector.

3. The method of claim 2, wherein the virus vector is selected from the group consisting of a virus vector construct, an RNA vector replicon, a DNA replicon plasmid, a eukaryotic layered vector initiation system, a virus vector particle, and a self-replicating virus-derived RNA molecule.

4. The method of claim 1, further comprising the step of introducing the virus vector into the packaging cell.

5. The method of claim 4, wherein the virus vector is introduced into the packaging cell by infection.

6. The method of claim 4, wherein the virus vector is introduced into the packaging cell by transfection.

7. The method of claim 6, wherein the packaging cell is transfected using a transfection reagent selected from the group consisting of a lipoplex, calcium phosphate, a liposome, polyethyleneimine (PEI), a cationic nanoemulsion, a lipid nanoparticle, protamine, polyarginine, polylysine, and a cationic lipid.

8. The method of claim 7, wherein the transfection reagent is a lipoplex and the lipoplex comprises 1:1 (w/w) 1,2-dioleoyl-3-trimethylammonium-propane (chloride salt) (DOTAP): 1,2-dioleoyl-sn-glycero-3-phosphoethanolamine (DOPE).

9. The method of claim 7, wherein the transfection reagent is PEI.

10. The method of claim 4, wherein the virus vector is introduced into the packaging cell by electroporation.

11. The method of claim 3, wherein the virus vector is the DNA replicon plasmid.

12. The method of claim 3, wherein the virus vector is a self-replicating virus-derived RNA molecule.

13. The method of claim 12, wherein the virus vector is introduced into the packaging cell by transfection of the cell with the self-replicating virus-derived RNA molecule.

14. The method of claim 12, wherein the virus vector is introduced into the packaging cell by transfection of the cell with a DNA molecule encoding the self-replicating virus-derived RNA molecule.

15. The method of claim 1, wherein the packaging cell is transfected or infected with a virus vector in suspension culture.

16. The method of claim 1, wherein the packaging cell is transfected or infected with a virus vector in adherent culture.

17. The method of claim 2, wherein the one or more virus structural protein expression cassettes is stably integrated in the packaging cell.

18. The method of claim 1, wherein the packaging cell is derived from a mammalian cell.

19. The method of claim 1, wherein the packaging cell is derived from an avian cell.

20. The method of claim 19, wherein the avian cell is a duck cell.

21. The method of claim 1, wherein the conditions comprise a temperature of about 32° C.

22. (canceled)

23. The method of claim 1, wherein the conditions comprise a temperature range from about 31° C. and about 34° C.

24. The method of claim 1, wherein the packaging cell is cultured at said temperature for at least 6 hours after introduction of a virus vector to the packaging cells.

25. The method of claim 1, wherein the packaging cell is cultured at said temperature for at least 12 hours after introduction of a virus vector to the packaging cell.

26. The method of claim 1, wherein the packaging cell is cultured at the temperature for at least 24 hours after introduction of a virus vector to the packaging cell.

27. The method of claim 1, wherein the packaging cell is cultured at the temperature in suspension culture.

28. The method of claim 1, wherein the packaging cell is cultured at the temperature in adherent culture.

29. The method of claim 1,
which further comprises one or more steps for separating the virus replicon particles from the packaging cell, whereby a composition substantially free of packaging cells and containing the virus replicon particles is produced.

30. The method according to claim 1, which further comprises one or more steps for purifying the virus replicon particles from the culture medium, whereby a purified composition containing the virus replicon particles is produced.

31. The method of claim 1, which further comprises one or more steps for formulating the virus replicon particles into a pharmaceutical composition, whereby a pharmaceutical composition containing the virus replicon particles is produced.

32. The method of claim 2, wherein the virus vector comprises a heterologous coding sequence encoding an antigen derived from a pathogen.

33. The method of claim 2, wherein the virus vector comprises a heterologous coding sequence encoding a glycoprotein.

34. The method of claim 1, wherein the virus replicon particles are alphavirus replicon particles.

35. The method of claim 34, wherein the packaging cell comprises (i) a first alphavirus structural protein expression cassette which directs expression of an alphavirus capsid protein; and (ii) a second alphavirus structural protein expression cassette which directs expression of at least one of an alphavirus E1 glycoprotein and an alphavirus E2 glycoprotein.

36. The method of claim 34, wherein the packaging cell comprises (i) a first alphavirus structural protein expression cassette which directs expression of an alphavirus capsid protein; and (ii) a second alphavirus structural protein expression cassette which directs expression of at least one of an alphavirus E1 glycoprotein and an alphavirus E2 glycoprotein but not the alphavirus capsid protein.

37. The method of claim 34, wherein the packaging cell comprises:
(a) one or more alphavirus structural protein expression cassettes directing expression of alphavirus structural proteins; and
(b) an alphavirus vector.

38. The method of claim 37, wherein the alphavirus vector is selected from the group consisting of an alphavirus vector construct, an RNA vector replicon, a DNA replicon plasmid, a eukaryotic layered vector initiation system, an alphavirus vector particle, and a self-replicating alphavirus-derived RNA molecule.

39. The method of claim 34, wherein at least 1×10⁶ IU/ml, at least 1×10⁷ IU/ml, or at least 1×10⁸ IU/ml, of alphavirus replicon particles are produced.

40. The method of claim 34, wherein the alphavirus replicon particles comprise a Venezuelan Equine Encephalitis (VEE) derived vector construct packaged with Sindbis (SIN) capsid and/or envelope glycoproteins.

41. The method of claim 34, wherein the number of alphavirus replicon particles produced in the method is at least 2-fold, at least 5-fold, or at least 10-fold, the number obtainable by performing a corresponding method in which the packaging cell is cultured at a temperature above about 35° C.


SEQUENCE LISTING (1 sequence)

SEQ ID NO: 1 - DNA, 12113 bp, Artificial Sequence
  plasmid CMV-TC83CR-GFP
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtagacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctcctcaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagagggatcacgggagaaaccgtgggatacgcggttacacacaatagcgagggcttcttgctatgcaaagttactgacacagtaaaaggagaacgggtatcgttccctgtgtgcacgtacatcccggccaccataaactcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtcgacgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataatctagacggcgcgcccacccagcggccgccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaaaaagggtcggcatggcatctccacctcctcgcggtccgacctgggcatccgaaggaggacgcacgtccactcggatggctaagggagagccacgtttgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctaattcatggttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggccggatcagcttatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctactgaacggtgatccccaccggaattgcggccggccgcggaatttcgactctaggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccg